(19) United States
(12) Patent Application Publication
(10) Pub. No.: US 2025/0312597 A1
(43) Pub. Date: Oct. 9, 2025

(54) SWALLOWING FUNCTION RECOVERY DEVICE AND SWALLOWING FUNCTION RECOVERY SYSTEM

(71) Applicant: Kazuyoshi SUZUKI, CHIBA (JP)

(72) Inventor: Kazuyoshi SUZUKI, CHIBA (JP)

(21) Appl. No.: 18/865,139

(22) PCT Filed: Aug. 31, 2023
(86) PCT No.: PCT/JP2023/031754

§ 371 Date: Nov. 12, 2024

(30) Foreign Application Priority Data
Aug. 31, 2022 (JP) 2022-148591

(51) Int. Cl. A61N 1/36; A61N 1/04; A61N 1/05

(52) U.S. Cl. CPC A61N 1/36003; A61N 1/0452; A61N 1/0548

(57) ABSTRACT

The present invention is configured to provide a device or the like that can efficiently recover functions of various muscles involved in swallowing function, in which an electrical connection state between a tongue (T) of a subject (animal such as human) (S) and a tongue guide (12) is maintained by a tongue interaction mechanism (11), an electrical connection state between an anterior throat portion (F) of the subject(S) and a pair of anterior throat portion guides (22) is maintained by an anterior throat portion interaction mechanism (21) in a direct or indirect manner, and accordingly, an EMS signal or an EMS current generated by an EMS signal generator (40) is reliably transmitted or flowed between the tongue (T) and the anterior throat portion (F) of the subject(S) through each of the tongue guide (12) and the pair of anterior throat portion guides (22).

### TECHNICAL FIELD

The present invention relates to a swallowing function recovery device and a swallowing function recovery system for recovering a function of a muscle of a part from the mouth to the pharynx, particularly for overcoming dysphagia by recovering the function.

### BACKGROUND ART

It is known that a disease such as cerebral infarction or aging causes a problem such as dysphagia. In a case of having dysphagia, there is a possibility that saliva, food, and/or gastric juice may enter the trachea. There is also a possibility that saliva enters the trachea unconsciously during sleeping. As a result, aspiration pneumonia may occur, but this is a terrible disease that is likely to lead to death for the elderly.

The function of the tongue muscle can be recovered by a movement such as sticking out the tongue, but for various muscles involved in the action of swallowing chewed food, it is not possible to build the muscle strength to swallow food without actually consuming food.

### SUMMARY OF INVENTION

### Technical Problem

However, since swallowing is caused by multiple nerves and muscles in the oral cavity, the pharynx, and the esophagus working in cooperation, the reality is that it is quite difficult to cause a person to eat when the person is unable to eat. The swallowing movement is divided into three phases, but particularly in the second phase, the tongue muscle, the oropharyngeal muscle, and the hypopharyngeal muscle work in cooperation, and there is no choice but to recover the function by actually moving these muscles. In addition, various muscles such as the digastric muscle posterior belly, the digastric muscle anterior belly, the geniohyoid muscle, the sternocleidomastoid muscle, the clavicular head, the masseter muscle, the trapezius muscle, the thyrohyoid muscle, the sternothyroid muscle, and the scalene muscle are also working.

Therefore, an object of the present invention is to provide a device or the like that can efficiently recover the functions of various muscles involved in the swallowing function.

### Solution to Problem

In order to solve the above-described problem, the present inventor has thought that, even when a person does not eat, a low-frequency current is allowed to flow to the anterior throat portion from the body surface or the outside, and accordingly, the muscle of the throat is contracted, and thus the muscle strength is recovered and enhanced. However, in a case where the low-frequency current is allowed to flow from the body surface to the anterior throat portion, there is a possibility that the action of the low-frequency current is limited to the periphery of the body surface. Therefore, the present inventor has thought that it may be possible to achieve the recovery and/or enhancement of muscle strength by flowing a low-frequency current from the tongue muscle to the throat muscle through the tongue inside the mouth to contract these muscles.

According to the present invention, there is provided a swallowing function recovery device including: a tongue guide; an anterior throat portion guide; a tongue interaction mechanism configured to maintain an electrical connection state between the tongue guide and a tongue of a subject; an anterior throat portion interaction mechanism configured to maintain an electrical connection state between the anterior throat portion guide and an anterior throat portion of the subject; and an EMS signal generator configured to transmit an EMS signal between the tongue guide and the anterior throat portion guide.

With the swallowing function recovery device having the above-described configuration, the tongue interaction mechanism maintains the electrical connection state between the tongue of the subject (animal such as a human) and the tongue guide, and the anterior throat portion interaction mechanism maintains the electrical connection state between the anterior throat portion of the subject and the anterior throat portion guide. The term “electrical connection state” means a state where an EMS signal or an EMS current can be transmitted or flowed to the subject through the guide. “EMS” means electrical muscle stimulation. As a result, the EMS signal or the EMS current generated by the EMS signal generator is reliably transmitted or flows between the tongue and the anterior throat portion of the subject through each of the tongue guide and the anterior throat portion guide. Due to the influence of the EMS signal, various muscles between the tongue and the anterior throat portion involved in the swallowing action are contracted, and accordingly, the muscle strength or function (muscle strength for swallowing an object of the subject) of the muscle is recovered or maintained.

In the swallowing function recovery device having the above-described configuration, preferably, a first tongue guide and a second tongue guide are provided as the tongue guide, a first anterior throat portion guide and a second anterior throat portion guide are provided as the anterior throat portion guide, and the EMS signal generator is configured to generate a first EMS signal and a second EMS signal having different frequencies, to transmit the first EMS signal between the first tongue guide and the first anterior throat portion guide, and to transmit the second EMS signal between the second tongue guide and the second anterior throat portion guide.

With the swallowing function recovery device having the above-described configuration, the first EMS signal flowing between the first tongue guide and the first anterior throat portion guide and the second EMS signal flowing between the second tongue guide and the second anterior throat portion guide and having a frequency different from that of the first EMS signal are caused to interfere with each other, whereby the muscle of the anterior throat portion is stimulated by the interference wave. In particular, a pulse current can flow to a muscle part having a large depth of the anterior throat portion. The interference wave stimulation is similarly applied to the tongue muscles.

In the swallowing function recovery device having the above-described configuration, preferably, the anterior throat portion interaction mechanism includes a neck wrapping member that is wrapped around a neck portion of the subject.

With the swallowing function recovery device having the above-described configuration, the neck wrapping member is wrapped around the neck, and accordingly, a state where the anterior throat portion guide is in contact with the anterior throat portion is reliably maintained. In a case where the anterior throat portion guide is provided to be displaceable in the neck wrapping member, the position of the anterior throat portion guide can be finely adjusted according to a difference in the thickness or the like of the neck of the subject. A fastener such as a buckle or a hook-and-loop fastener may be provided on the neck wrapping member (for example, a belt).

In the swallowing function recovery device having the above-described configuration, preferably, the anterior throat portion interaction mechanism includes a neck hanging member that is hung from a rear portion of a neck portion to the anterior throat portion of the subject.

With the swallowing function recovery device having the above-described configuration, the anterior throat portion guide provided in the neck hanging member is maintained in a state of abutting against the anterior throat portion by banging the neck hanging member (clip or neck band) from the rear side to the front side of the neck portion. The neck hanging member has an advantage that it is easy to attach and detach the neck hanging member to and from the neck portion or the anterior throat portion.

In the swallowing function recovery device having the above-described configuration, preferably, the anterior throat portion interaction mechanism includes an adhesive pad configured to be attachable to and detachable from the anterior throat portion of the subject.

With the swallowing function recovery device having the above-described configuration, a state where the anterior throat portion guide is in contact with (the skin of) the anterior throat portion of the subject can be reliably maintained by the adhesive pad. The adhesive pad is attachable to and detachable from the skin of the anterior throat portion.

In the swallowing function recovery device having the above-described configuration, preferably, the anterior throat portion interaction mechanism includes a suction cup that is attachable to and detachable from the anterior throat portion of the subject.

With the swallowing function recovery device having the above-described configuration, the guide can be suctioned to the skin of the throat due to a special structure of a surface that touches the anterior throat portion, which is called a suction cup. The suction cup is freely attachable to and detachable from the skin.

In the swallowing function recovery device having the above-described configuration, preferably, the anterior throat portion interaction mechanism includes a base portion, and a pair of extension portions that extends from the base portion in a bifurcated manner to sandwich the anterior throat portion of the subject from left and right, and the anterior throat portion guide is provided on each of the pair of extension portions.

With the swallowing function recovery device having the above-described configuration, a state where the anterior throat portion guides provided on each of the pair of extension portions are in contact with the left side and the right side of the anterior throat portion is reliably maintained by using the pair of extension portions that extends from the base portion in a bifurcated manner to sandwich the anterior throat portion of the subject from the left and right. The strength of the contact pressure of the anterior throat portion guide on the anterior throat portion and the contact point of the anterior throat portion guide on the anterior throat portion can be adjusted, for example, by moving the base portion. In a case where the anterior throat portion guide is configured with a roller, the anterior throat portion guide can be brought into contact with the muscle constituting the anterior throat portion while rolling the anterior throat portion guide along the muscle. This can make the subject feel the sensation of training the swallowing function.

In the swallowing function recovery device having the above-described configuration, preferably, the tongue interaction mechanism includes a tongue depressor.

With the swallowing function recovery device having the above-described configuration, the tongue depressor, which is a tool for holding the tongue in a case of examining the oral cavity and/or the esophagus, is used, and accordingly, a state where the tongue guide is in contact with the tongue can be reliably maintained. In general, a stainless steel tongue depressor that is used while managing sterilization and disinfection is used as the tongue depressor. A disposable wooden tongue depressor may be used.

In the swallowing function recovery device having the above-described configuration, preferably, the tongue interaction mechanism includes a finger cot.

With the swallowing function recovery device having the above-described configuration, the finger cot worn on the finger of the subject or a third party is used, and accordingly, a state where the tongue guide is in contact with the tongue can be reliably maintained.

In the swallowing function recovery device having the above-described configuration, preferably, the tongue interaction mechanism includes a suction cup that is attachable to and detachable from the tongue.

With the swallowing function recovery device having the above-described configuration, the suction cup is easily suctioned to the tongue wet with saliva, and the state where the tongue guide is in contact with the tongue of the subject can be reliably maintained. In addition, the suction cup is easily removed from the tongue of the subject without particular difficulty.

### BRIEF DESCRIPTION OF DRAWINGS

FIG. 1 is a view illustrating a configuration of a swallowing function recovery device according to a first embodiment of the present invention.

FIG. 2 is a view illustrating a configuration of a tongue interaction mechanism according to the first embodiment.

FIG. 3 is a view illustrating a configuration of an anterior throat portion interaction mechanism according to the first embodiment.

FIG. 4 is a view illustrating a configuration of a swallowing function recovery device according to a second embodiment of the present invention.

FIG. 5 is a view illustrating a configuration of a tongue interaction mechanism according to the second embodiment.

FIG. 6 is a view illustrating a configuration of an anterior throat portion interaction mechanism according to the second embodiment.

FIG. 7 is a view illustrating a configuration of a tongue interaction mechanism according to another embodiment.

FIG. 8 is a view illustrating a configuration of a tongue interaction mechanism according to another embodiment.

FIG. 9 is a view illustrating a configuration of an anterior throat portion interaction mechanism according to another embodiment.

FIG. 10 is a view illustrating a configuration of an anterior throat portion interaction mechanism according to another embodiment.

FIG. 11 is a view illustrating a configuration of an anterior throat portion interaction mechanism according to another embodiment.

FIG. 12 is a view illustrating a configuration of an anterior throat portion interaction mechanism according to another embodiment.

### DESCRIPTION OF EMBODIMENTS

### First Embodiment

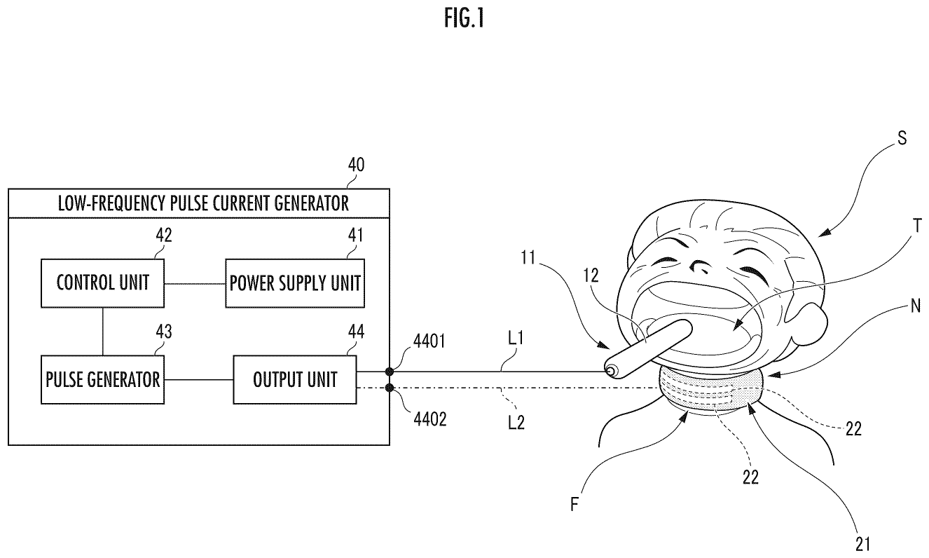

A swallowing function recovery device according to a first embodiment of the present invention illustrated in FIG. 1 includes a tongue guide 12, a pair of anterior throat portion guides 22, a tongue interaction mechanism 11, an anterior throat portion interaction mechanism 21, and an EMS signal generator 40.

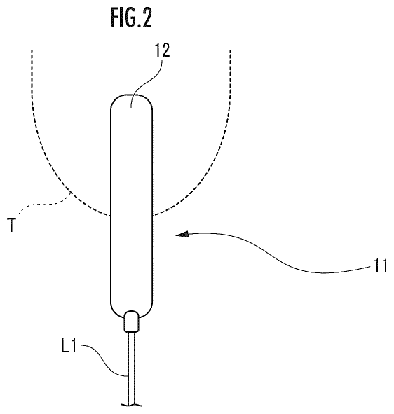

As illustrated in FIGS. 1 and 2, the tongue interaction mechanism 11 includes a tongue depressor. As illustrated in FIG. 2, at least a part of the tongue depressor is composed of a conductive material such as stainless steel, and the tongue guide 12 is composed of the conductive material. As the tongue guide 12, an electrode that rarely gives a tingling sensation and is preferably made of conductive polyester may be employed.

The tongue depressor may be configured with an insulating material such as wood or synthetic resin, and a part thereof may be configured with a conductive material. The tongue guide 12 may be composed of a metal clip and may be removably attached to the tongue depressor. The tongue depressor is pressed against a tongue T of a subject S, and accordingly, the electrical connection state between the tongue guide 12 and the tongue T is maintained. The shape and size of the tongue depressor may be adjusted such that the electrical connection state between the tongue guide 12 and the tongue T is maintained as the subject S holds the tongue depressor.

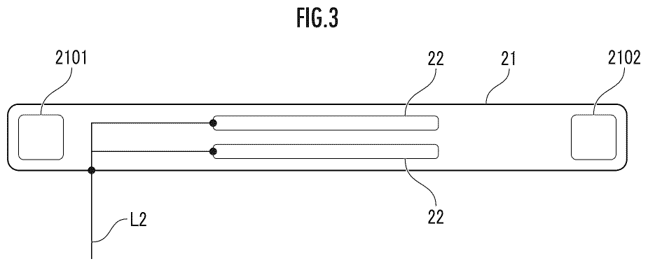

As illustrated in FIGS. 1 and 3, the anterior throat portion interaction mechanism 21 is composed of a belt as a neck wrapping member. The belt is made of, for example, a leather material, a natural material fiber such as cotton, a synthetic fiber such as polyester, or the like. As illustrated in FIG. 3, each of a male hook-and-loop fastener 2101 and a female hook-and-loop fastener 2102 are provided at each of both end portions of the belt. Instead of a hook-and-loop fastener, a connection mechanism such as a buckle, a hook, or a snap button may be provided for connecting both end portions (or one end portion and an intermediate portion) of the belt.

The pair of substantially rectangular strip-shaped anterior throat portion guides 22 is spaced apart from each other in a width direction (short direction) of the belt on a surface or inside of the belt, and are disposed to extend parallel to a longitudinal direction of the belt. As the pair of the anterior throat portion guides 22, an electrode that rarely gives a tingling sensation to the subject S and is preferably made of conductive polyester may be employed. Each of the number, shape, size, and disposition mode of the anterior throat portion guides 22 may be variously changed. The belt is wrapped around a neck portion N of the subject S, and both end portions are connected to each other, and accordingly, an electrical connection state between the anterior throat portion F of the subject S and each of the pair of first anterior throat portion guides 221 and the pair of second anterior throat portion guides 222 is maintained.

As illustrated in FIG. 1, the EMS signal generator 40 includes a power supply unit 41, a control unit 42, a pulse generator 43, and an output unit 44. The power supply unit 41 is configured to supply electric power to the control unit 42 and the like. The control unit 42 is configured by a computer, and a computing processing device (processor) is configured to read out necessary software (program) and data from a storage device (memory) and to execute predetermined computing processing according to the software based on the data. The pulse generator 43 is configured to generate the EMS signal as a pseudo waveform signal by oscillating a pulse signal and adjusting a period and/or an amplitude of the pulse signal in response to a command from the control unit 42. The EMS signal is composed of, for example, at least one of a low-frequency electrical signal (20 to 100 Hz), a medium-frequency electrical signal (1,000 to 10,000 Hz), or a high-frequency electrical signal (10,000 to 20,000 Hz). The output unit 44 is connected to the tongue guide 12 through a terminal 4401 and a conductive wire L1, and is connected to each of the pair of anterior throat portion guides 22 through a terminal 4402 and a conductive wire L2.

### Effects of Operation

According to the swallowing function recovery device having the above-described configuration, the tongue interaction mechanism 11 maintains the electrical connection state between the tongue T of the subject S (animal such as a human) and the tongue guide 12, and the anterior throat portion interaction mechanism 21 maintains the electrical connection state between the anterior throat portion F of the subject S and the pair of anterior throat portion guide 22 in a direct or indirect manner. As a result, the EMS signal or the EMS current generated by the EMS signal generator 40 is reliably transmitted or flows between the tongue T and the anterior throat portion F of the subject S through the tongue guide 12 and each of the pair of anterior throat portion guides 22. Due to the influence of the EMS signal, various muscles between the tongue T and the anterior throat portion F involved in the swallowing action are contracted, and accordingly, the muscle strength or function (muscle strength for swallowing an object of the subject S) of the muscle is recovered or maintained.

The application of the EMS signal to the tongue T and the anterior throat portion F can be performed even while the subject S is watching television or reading a book, and it is possible to recover or train the muscle strength of the muscle involved in the swallowing function both at home and while away. The control unit 42 may detect that the subject S has pressed a switch of a remote controller of an electric appliance such as a television, an air conditioner, a personal computer, and an audio device through wireless communication between the remote controller and the EMS signal generator 40, and the EMS signal may be transmitted between the tongue T and the anterior throat portion F of the subject S in response to the detection.

### Second Embodiment

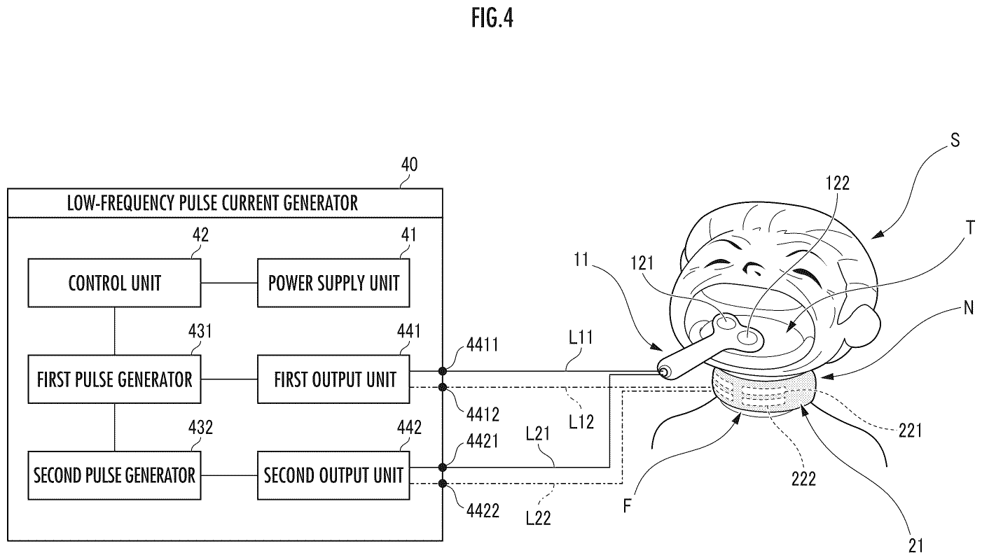

A swallowing function recovery device according to a second embodiment of the present invention illustrated in FIG. 4 includes a first tongue guide 121, a second tongue guide 122, the pair of first anterior throat portion guides 221, the pair of second anterior throat portion guides 222, the tongue interaction mechanism 11, the anterior throat portion interaction mechanism 21, and the EMS signal generator 40. Regarding the second embodiment, differences from the first embodiment will be described, and the same reference numerals will be used for the same configurations as those in the first embodiment, and the description thereof will be appropriately omitted.

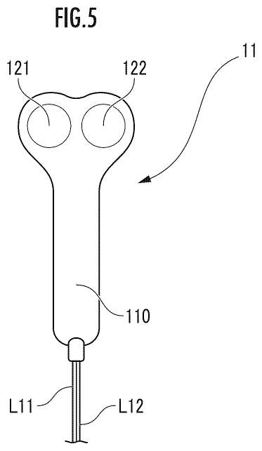

As illustrated in FIGS. 4 and 5, the tongue interaction mechanism 11 includes a tongue depressor. As illustrated in FIG. 5, the first tongue guide 121 and the second tongue guide 122, which are composed of a conductive material, are provided at a tip end portion of a base (the tip end portion thereof is formed in an elongated shape to be slightly wide) of a tongue depressor composed of an insulating material such as wood or a synthetic resin. The first tongue guide 121 and the second tongue guide 122 may be composed of a metal clip and may be removably attached to the tongue depressor. By pressing the tongue depressor against the tongue T of the subject S, the electrical connection state between the first tongue guide 121, the second tongue guide 122, and the tongue T is maintained. The shape and size of the tongue depressor may be adjusted such that the electrical connection state between the first tongue guide 121 and the second tongue guide 122 and the tongue T is maintained as the subject S holds the tongue depressor.

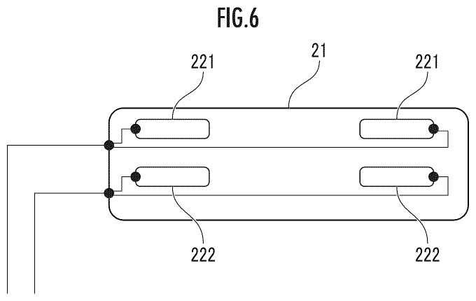

The anterior throat portion interaction mechanism 21 is configured by a single adhesive pad or an adhesive sheet. As illustrated in FIG. 6, a pair of substantially rectangular first anterior throat portion guides 221 are disposed on the surface or the inner side of the belt to be spaced apart from each other in the longitudinal direction of the adhesive pad. As illustrated in FIG. 6, a pair of substantially rectangular second anterior throat portion guides 222 are disposed on the surface or the inner side of the adhesive pad to be spaced apart from each other in the longitudinal direction of the adhesive pad. In the width direction (short direction) of the adhesive pad, each of the pair of first anterior throat portion guides 221 and each of the pair of second anterior throat portion guides 222 are disposed to be spaced from each other. Each of the number, shape, size, and disposition mode of the first anterior throat portion guide 221 and/or the second anterior throat portion guide 222 may be variously changed. The anterior throat portion interaction mechanism 21 may be configured by a plurality of adhesive pads or adhesive sheets, and one or a plurality of anterior throat portion guides may be provided on each adhesive pad. By bonding the adhesive pad to the anterior throat portion F of the subject S, a state where the EMS signal can be transmitted to the anterior throat portion F of the subject S through the pair of first anterior throat portion guides 221 and the pair of second anterior throat portion guides 222 is maintained.

As in the first embodiment, the anterior throat portion interaction mechanism 21 may be configured by a belt as a neck wrapping member (refer to FIG. 3). In this case, the first anterior throat portion guide 221 and the second anterior throat portion guide 222 may be disposed on the inner surface of the belt or may be incorporated therein.

As illustrated in FIG. 4, the EMS signal generator 40 includes the power supply unit 41, the control unit 42, a first pulse generator 431, a second pulse generator 432, a first output unit 441, and a second output unit 442. The first pulse generator 431 is configured to generate the first EMS signal as a pseudo waveform signal by generating a pulse signal and adjusting a period and/or an amplitude of the pulse signal in response to a command from the control unit 42. The second pulse generator 432 is configured to generate the second EMS signal as a pseudo waveform signal by generating a pulse signal and adjusting a period and/or an amplitude of the pulse signal in response to a command from the control unit 42. The first EMS signal and the second EMS signal have different frequencies. For example, the first EMS signal may be a low-frequency electrical signal (20 to 100 Hz), and the second EMS signal may be a medium-frequency electrical signal (1,000 to 10,000 Hz) or a high-frequency electrical signal (10,000 to 20,000 Hz). The first output unit 441 is connected to the first tongue guide 121 through a terminal 4411 and the conductive wire L11, and is connected to each of the pair of first anterior throat portion guides 221 through a terminal 4412 and a conductive wire L21. The second output unit 442 is connected to the second tongue guide 122 through a terminal 4421 and a conductive wire L12, and is connected to each of the pair of second anterior throat portion guides 222 through a terminal 4422 and a conductive wire L22.

### Effects of Operation

With the swallowing function recovery device having the above-described configuration, the tongue interaction mechanism 11 maintains the electrical connection state between the tongue T of the subject S (animal such as human) and the tongue guide 12, and the anterior throat portion interaction mechanism 21 maintains the electrical connection state between the anterior throat portion F of the subject S and the pair of first anterior throat portion guides 221 and the pair of second anterior throat portion guides 222 in a direct or indirect manner. Accordingly, the first EMS signal flowing between the first tongue guide 121 and the first anterior throat portion guide 221, and the second EMS signal flowing between the second tongue guide 122 and the second anterior throat portion guide 222 and having a frequency different from that of the first EMS signal, which are generated by the EMS signal generator 40, are caused to interfere with each other, whereby the muscle of the anterior throat portion F and the tongue T is stimulated by the interference wave. In particular, a pulse current can flow to a muscle part having a large depth of the anterior throat portion F. Due to the influence of the interference wave, various muscles between the tongue T and the anterior throat portion F involved in the swallowing action are contracted, and accordingly, the muscle strength of the muscle or the recovery or maintenance of the swallowing function is achieved.

### Other Embodiments of Present Invention

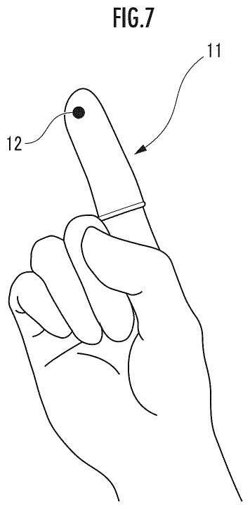

As illustrated in FIG. 7, the tongue interaction mechanism 11 may be configured with a finger cot. One or a plurality of tongue guides 12 are provided on an outer surface, an inside, or an inner surface of a tip end portion (a part corresponding to a distal phalanx of a finger) of the finger cot. The finger cot is mounted on the finger (for example, the index fingers) of the subject S or the third party such that the tongue guide 12 is on the ventral side, the ventral side of the tip end portion of the finger cot is pressed against the tongue T of the subject S, and accordingly, a state where the tongue guide 12 is electrically connected to the tongue T can be reliably maintained.

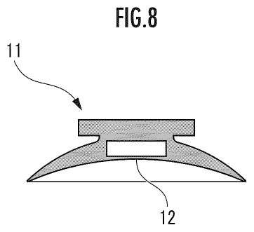

As illustrated in FIG. 8, the tongue interaction mechanism 11 may be configured by one or a plurality of suction cups. One or a plurality of tongue guides 12 are provided on a surface or the inside of the suction cup. By the suction cup being suctioned to the tongue T of the subject S, a state where the tongue guide 12 is electrically connected to the tongue T can be reliably maintained.

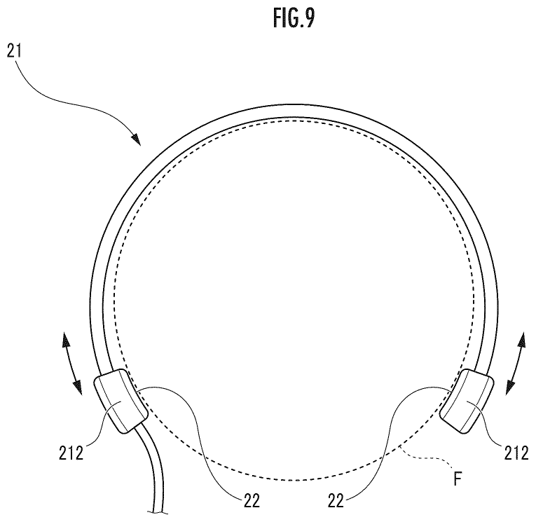

As illustrated in FIG. 9, the anterior throat portion interaction mechanism 21 may be formed of a neck hanging member or a clip that is wound from the rear side to the front side of the neck portion and is partially open. The neck hanging member is made of an elastic member such as a synthetic resin. The neck hanging member may be configured of a plurality of links connected by one or a plurality of joint mechanisms, and may be configured such that a relative posture of a pair of links connected by the joint mechanism can be changed by adjusting a bending angle of the joint mechanism. Pressing members 212 are provided at each of a pair of end portions of the neck hanging member. The pressing member 212 may be composed of a soft member such as sponge or silicone rubber or an elastic member. The anterior throat portion guide 22 is provided on a surface or the inside of each of the pair of pressing members 212 on the anterior throat portion F side. As indicated by an arrow in FIG. 9, the pair of left and right pressing members 212 may be configured to be slidable along the neck hanging member. Since the neck hanging member is formed of the elastic member, the pressing member 212 can be reliably brought into contact with the anterior throat portion F of the subject S. The pressing member 212 may be omitted, and one or a plurality of anterior throat portion guides 22 may be provided on the surface or the inside of the neck hanging member.

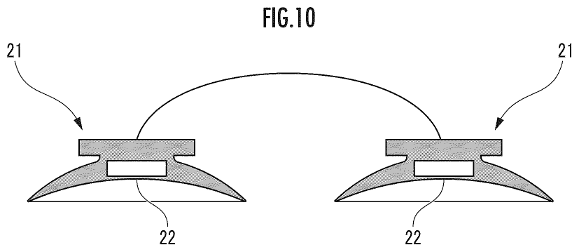

As illustrated in FIG. 10, the anterior throat portion interaction mechanism 21 may be composed of one or a plurality of suction cups. One or a plurality of anterior throat portion guides 22 are provided on a surface or the inside of the suction cup. The plurality of suction cups may be independent of each other or may be connected to each other by a connecting member. The neck hanging member constitutes the connecting member in the embodiment of FIG. 9, and one or a plurality of suction cups may be provided at each of both end portions of the neck hanging member as the anterior throat portion interaction mechanism 21. By the suction cup being suctioned to the anterior throat portion F of the subject S, a state where the anterior throat portion guide 22 is electrically connected to the anterior throat portion F can be reliably maintained.

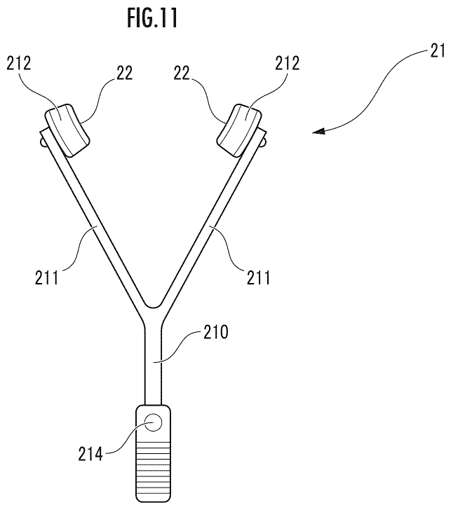

As illustrated in FIG. 11, the anterior throat portion interaction mechanism 21 may include a base portion 210 and a pair of extension portions 211 that extends from the tip end portion of the base portion 210 in a bifurcated manner to sandwich the anterior throat portion F of the subject S from left and right. At least one pair of the extension portions 211 of the anterior throat portion interaction mechanism 21 is configured to have elasticity by a material such as a synthetic resin or a metal. Each of the pair of extension portions 211 is provided with the pressing member 212. The pressing member 212 may be composed of a soft member such as sponge or silicone rubber or an elastic member. The anterior throat portion guide 22 is provided on a surface or the inside of each of the pair of pressing members 212 on the anterior throat portion F side. A switch 214 for switching the operation of the EMS signal generator 40 between ON and OFF is provided at the base portion 210. The EMS signal generator 40 may be incorporated in the base portion 210.

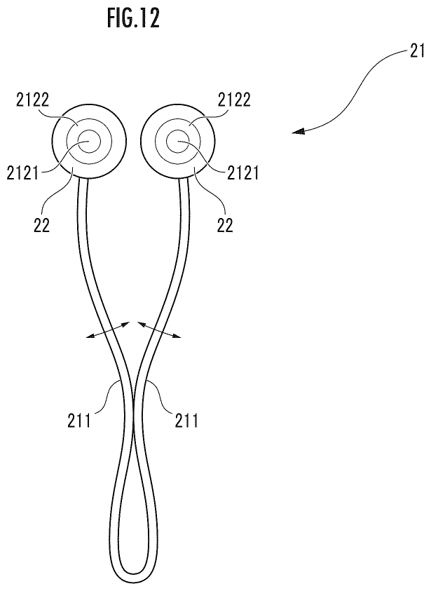

As illustrated in FIG. 12, the anterior throat portion interaction mechanism 21 may include the base portion 210 and the pair of extension portions 211 that extends from the tip end portion of the base portion 210 in a bifurcated manner to sandwich the anterior throat portion F of the subject S from left and right. At least one pair of the extension portions 211 of the anterior throat portion interaction mechanism 21 is configured to have elasticity by a material such as a synthetic resin or a metal. Each of the pair of extension portions 211 is provided with the pressing member 212. The pressing member 212 is composed of a rotational movement shaft 2121 and a roller 2122 that is substantially cylindrical or substantially columnar and rotates around the rotational movement shaft 2121. The roller 2122 may be formed in a substantially spherical shape or a substantially polyhedral shape. The roller 2122 may be composed of a hard member such as a synthetic resin, ceramics, and/or metal. The anterior throat portion guide 22 having a substantially cylindrical shape is provided along the outer periphery of the roller 2122. The plurality of anterior throat portion guides 22 may be disposed to be spaced from each other along the outer periphery of the roller 2122.

### INDUSTRIAL AVAILABILITY

The swallowing function recovery device according to the present invention can recover the functions of various muscles involved in the swallowing function of the subject using the EMS signal, which contributes to the industry. The subject can be extended not only to a human but also to an animal such as a dog and a cat.

### DESCRIPTION OF REFERENCE NUMERALS

11 tongue interaction mechanism
12 tongue guide
121 first tongue guide
122 second tongue guide
21 anterior throat portion interaction mechanism
22 anterior throat portion guide
221 first anterior throat portion guide
222 second anterior throat portion guide
40 EMS signal generator
41 power supply unit
42 control unit
431 first pulse generator
432 second pulse generator
44 output unit
441 first output unit
442 second output unit
F anterior throat portion
N neck portion
S subject
T tongue

1. A swallowing function recovery device comprising:
a tongue guide;
an anterior throat portion guide;
a tongue interaction mechanism configured to maintain an electrical connection state between the tongue guide and a tongue of a subject;
an anterior throat portion interaction mechanism configured to maintain an electrical connection state between the anterior throat portion guide and an anterior throat portion of the subject; and
an EMS signal generator configured to transmit an EMS signal between the tongue guide and the anterior throat portion guide.

2. The swallowing function recovery device according to claim 1, wherein
a first tongue guide and a second tongue guide are provided as the tongue guide,
a first anterior throat portion guide and a second anterior throat portion guide are provided as the anterior throat portion guide, and
the EMS signal generator is configured to generate a first EMS signal and a second EMS signal having different frequencies, to transmit the first EMS signal between the first tongue guide and the first anterior throat portion guide, and to transmit the second EMS signal between the second tongue guide and the second anterior throat portion guide.

3. The swallowing function recovery device according to claim 1, wherein
the anterior throat portion interaction mechanism includes a neck wrapping member that is wrapped around a neck portion of the subject.

4. The swallowing function recovery device according to claim 1, wherein
the anterior throat portion interaction mechanism includes a neck hanging member that is hung from a rear portion of the neck portion to the anterior throat portion of the subject.

5. The swallowing function recovery device according to claim 1, wherein
the anterior throat portion interaction mechanism includes an adhesive pad configured to be attachable to and detachable from the anterior throat portion of the subject.

6. The swallowing function recovery device according to claim 1, wherein
the anterior throat portion interaction mechanism includes a suction cup that is attachable to and detachable from the anterior throat portion of the subject.

7. The swallowing function recovery device according to claim 1, wherein
the anterior throat portion interaction mechanism includes a base portion, and a pair of extension portions that extends from the base portion in a bifurcated manner to sandwich the anterior throat portion of the subject from left and right, and
the anterior throat portion guide is provided on each of the pair of extension portions.

8. The swallowing function recovery device according to claim 1, wherein
the tongue interaction mechanism includes a tongue depressor.

9. The swallowing function recovery device according to claim 1, wherein
the tongue interaction mechanism includes a finger cot.

10. The swallowing function recovery device according to claim 1, wherein
the tongue interaction mechanism includes a suction cup that is attachable to and detachable from the tongue.

11. A swallowing function recovery system comprising:
a tongue guide;
an anterior throat portion guide;
a tongue interaction mechanism configured to maintain a state where an EMS signal is applied to a tongue of a subject through the tongue guide;
an anterior throat portion interaction mechanism configured to maintain a state where the EMS signal is applied to an anterior throat portion of the subject through the anterior throat portion guide; and
an EMS signal generator, wherein
the EMS signal generated by the EMS signal generator is configured to be transmitted between the tongue guide and the anterior throat portion guide.

12. The swallowing function recovery system according to claim 11, wherein
a first tongue guide and a second tongue guide are provided as the tongue guide,
a first anterior throat portion guide and a second anterior throat portion guide are provided as the anterior throat portion guide, and
the EMS signal generator is configured to generate a first EMS signal and a second EMS signal having different frequencies, to transmit the first EMS signal between the first tongue guide and the first anterior throat portion guide, and to allow the second EMS signal to flow between the second tongue guide and the second anterior throat portion guide.
